(19) United States
(12) Patent Application Publication
(10) Pub. No.: US 2011/0251561 A1
(43) Pub. Date: Oct. 13, 2011

(54) TRANSDERMAL ADMINISTRATION DEVICE

(75) Inventors: Akinori Inou, Aichi (JP); Masahiro Takigawa, Kyoto (JP); Toru Sekiguchi, Tokyo (JP); Kaname Nakahara, Tokyo (JP); Toru Watanabe, Tokyo (JP)

(73) Assignees: LINTEC CORPORATION, Tokyo (JP); NANBU PLASTICS CO., LTD., Shizuoka (JP)

(21) Appl. No.: 13/055,378

(22) PCT Filed: Jul. 27, 2009
(86) PCT No.: PCT/JP09/63895

§ 371 Date: Jun. 23, 2011

(30) Foreign Application Priority Data
Jul. 25, 2008 (JP) 2008-191960

(51) Int. Cl. A61M 5/00; A61M 5/32

(52) U.S. Cl. 604/173

(57) ABSTRACT

The transdermal administration device of the present invention includes: a device body including a needle form body, which includes a plurality of microneedles formed on a surface thereof and is provided with a fluid passage hole through the front surface to a back surface thereof, and a medical fluid filling section for providing a medical fluid to the back surface of the needle form body; a stratum corneum peeling system; and a connection section for connecting the device body and the stratum corneum peeling system,
where the stratum corneum peeling system is connected to the device body by the connection section such that the microneedles of the needle form body of the device body are positioned in an area of a skin surface in which a stratum corneum is peeled by the stratum corneum peeling system.

### TECHNICAL FIELD

The present invention relates to a transdermal administration device for transdermally administering a medical fluid.

### BACKGROUND ART

An injection is conventionally used for administering a medical fluid or a medicine (referred to as a medical fluid, hereinafter) that cannot be administered orally. However, a method using an injection has a problem of providing a large degree of stress to a body, and further, of causing pain. Furthermore, a method for transdermal administration uses a medical patch containing a medical fluid; however, this method has a problem of requiring an extended period of time until the medicine takes effect and of limiting the kind of medical fluids usable.

In order to solve the problems, techniques called microneedle and microblade are recently under development.

A medical fluid administering method using a microneedle (Japanese Laid-Open Publication No. 2006-149818, Reference 1) is used for the purpose of reducing the time until the effect of the medicine appears by administering a medical fluid directly to a live cell layer (epidermis below the prickle cell layer) of the cuticle. The method is also used for the purpose of reducing a time until the effect of medicines, such as insulin and anesthetic, appears and a time until the effect of a physiological active substance, such as DNA, RNA, protein and peptide, appears, with a similar principle.

The microneedle is a microscopic needle with generally a tip of 10 to 50 μm in diameter, 100 to 200 μm in height, and with a base of 100 to 200 μm in diameter. The microneedle is a micro structure with a large aspect ratio (height/base diameter). Therefore, the medical fluid administering method using a microneedle has the advantage of not causing pain upon inserting it into a human body because the microneedle does not reach a pain spot though it penetrates a stratum corneum of a skin. As described above, the conventional method using the microneedle puts emphasis on relieving the stress to the body by the syringe, and in particular, on relieving pain due to the penetration of the needle.

Although the microneedle is extremely microscopic as described above, the microneedle, however, must penetrate the stratum corneum of the skin first to administer the medical fluid. For that reason, a sufficient length (or height) is required for the microneedle to penetrate the stratum corneum. Moreover, the microneedle is required to be sharp enough to penetrate the stratum corneum because the stratum corneum is not only relatively hard but also elastic. However, when both the length and sharpness are required at the same time, the microneedle will not have sufficient strength. Since there is a case where the microneedle snaps or breaks within a living body during the administration of a medical fluid, it is necessary that the microneedle does not remain within the body. Thus, it is not easy to manufacture a microneedle that is long and sharp and has a sufficient strength.

Japanese Laid-Open Publication No. 2005-334594 (Reference 2) proposes a medicine administering patch, where the medicine administering patch having carbon nanotube protrusions is pressed on the skin to transdermally transport the medicine after stratum corneum of a skin is peeled off using a pressure sensitive adhesive and the like, and the condition of the surface of the skin is made even and stable.

However, according to this method, the medicine administering patch having protrusions is pressed on the skin after keratin of the skin is peeled off using a pressure sensitive adhesive and the like. Such an administering operation is inconvenient and it is required to accurately position the point where the medicine is administered.
Reference 1: Japanese Laid-Open Publication No. 2006-149818
Reference 2: Japanese Laid-Open Publication No. 2005-334594

### SUMMARY OF INVENTION

The present invention is intended to solve the conventional problems described above. The objective of the present invention is to provide a transdermal administration device for securely transdermally administering a medical fluid even if the microneedles are short in length.

Another objective of the present invention is to provide a transdermal administration device capable of transdermally administering a medical fluid with an easy operation and securely transdermally administering a medical fluid to an area of skin surface where the stratum corneum has been peeled off.

The present invention is characterized as follows in order to solve the problems described above.

A transdermal administration device according to the present invention includes: a device body including a needle form body, which includes a plurality of microneedles formed on a surface thereof and is provided with a fluid passage hole through the front surface to a back surface thereof, and a medical fluid filling section for providing a medical fluid to the back surface of the needle form body; a stratum corneum peeling system; and a connection section for connecting the device body and the stratum corneum peeling system, wherein the stratum corneum peeling system is connected to the device body by the connection section such that the microneedles of the needle form body of the device body are positioned in an area of a skin surface in which a stratum corneum is peeled by the stratum corneum peeling system.

In one embodiment, the device body includes: a cylinder body in which a syringe filled with the medical fluid is movably inserted; and the needle form body attached to a tip portion of the syringe.

In one embodiment, a skin enlarging section for enlarging the skin when the cylinder body is pressed against the skin, is provided at a lower end opening of the cylinder body.

In one embodiment, the device body includes: a base sheet; a capsule-shaped medical fluid filling section provided on one surface of the base sheet; and the needle form body provided on the other surface of the base sheet; and a fragile portion is formed in the base sheet, the fragile portion being breakable by a pressure created by pressing the medical fluid filling section.

In one embodiment, the connection section includes a supporting sheet provided on the side of the needle form body of the device body; the supporting sheet includes an opening formed therein, the opening corresponding to the needle form body; and the stratum corneum peeling system is connected to the device body with the supporting sheet interposed therebetween.

In one embodiment, the connection section further includes a tearing sheet provided between the supporting sheet and the stratum corneum peeling system, and the tearing sheet can be torn along the skin surface so that it becomes possible to simultaneously tear the tearing sheet and the stratum corneum peeling system.

In one embodiment, the tearing sheet is constituted of: a first sheet positioned in the middle; and second and third sheets respectively positioned on both sides of the first sheet; the first sheet is adhered to end portions of the other two sheets, the end portions being folded; and the second sheet is connected to the supporting sheet and the third sheet is connected to a substrate sheet of the stratum corneum peeling system.

In one embodiment, the tearing sheet is constituted of: a second sheet with an end portion being folded; and a third sheet with an end portion being folded; a folding section of the second sheet is connected to the supporting sheet; and a folding section of the third sheet also serves as a substrate sheet of the stratum corneum peeling system.

In one embodiment, the tearing sheet includes: a first sheet positioned in the middle; and second and third sheets respectively positioned on both sides of the first sheet; an end portion of each of the sheets is folded; the first, second and third sheets are adhered to the other sheets at the folded portions; and the third sheet is connected to the stratum corneum peeling system.

In one embodiment, the tearing sheet includes: a long-length sheet; and a short-length sheet; the long-length sheet is folded and the folded portion is adhered with one side of the folded short-length sheet; and the other side of the short-length sheet is connected to the stratum corneum peeling system.

In one embodiment, a pressure sensitive adhesive layer is provided at least at a part of a side contacting the skin of the supporting sheet.

In one embodiment, the stratum corneum peeling system includes: a substrate sheet; and a pressure sensitive adhesive layer laminated on the substrate sheet, and the stratum corneum on the skin surface can be peeled by the pressure sensitive adhesive layer.

A transdermal administration device according to the present invention includes: a device body including a needle form body, which includes a plurality of microneedles formed on a surface thereof and is provided with a fluid passage hole through the front surface to a back surface thereof, and a medical fluid filling section for providing a medical fluid to a back surface of the needle form body; a stratum corneum peeling system including: a substrate sheet; and a pressure sensitive adhesive layer laminated on the substrate sheet, the pressure sensitive adhesive layer peeling off a stratum corneum of a skin surface; and a positioning sheet for connecting the device body and the stratum corneum peeling system, wherein the positioning sheet includes an opening, the device body is connected to one end portion of the positioning sheet such that the device body can be folded and piled on the positioning sheet, and the stratum corneum peeling system is connected to the other end portion of the positioning sheet such that the stratum corneum peeling system can be folded and piled on the positioning sheet, and wherein the microneedles of the needle form body are configured to be positioned at the opening when the device body is folded and piled on the positioning sheet, and the pressure sensitive adhesive layer of the stratum corneum peeling system is configured to be positioned at the opening when the stratum corneum peeling system is folded and piled on the positioning sheet.

In one embodiment, the positioning sheet is formed in a quadrilateral shape in a planar view, the device body is connected to one side of the positioning sheet, and the stratum corneum peeling system is connected to the other side of the positioning sheet.

In one embodiment, the positioning sheet is formed in a quadrilateral shape in a planar view, the device body is connected to one side of the positioning sheet, and the stratum corneum peeling system is connected to each of the other three sides of the positioning sheet.

In one embodiment, an extension piece is extended from the positioning sheet, and the device body is adhered on the extension piece.

In one embodiment, an extension piece is extended from the positioning sheet; the device body includes: the extension piece; the capsule-shaped medical fluid filling section provided on one surface of the extension piece; and the needle form body provided on the other surface of the extension piece; and the extension piece is provided with a fragile portion formed therein, the fragile portion being breakable by a pressure created by pressing the medical fluid filling section.

In one embodiment, the device body includes: a medical fluid filling section for housing the medical fluid; and the needle form body provided on the exterior surface of the medical fluid filling section; the plurality of microneedles are formed on the external surface of the needle form body; the fluid passage hole is provided in the needle form body through the front surface to a back surface thereof; and the fragile portion is provided in the medical fluid filling section, where the medical fluid filling section can be broken so that the medical fluid therein is provided to the back surface of the needle form body.

In one embodiment, the pressure sensitive adhesive layer is provided at least at a part of a side contacting the skin of the positioning sheet.

According to the present invention, the following effects can be achieved.

According to the present invention, the stratum corneum peeling system is connected to the device body by the connection section such that the microneedles of the needle form body of the device body are positioned in an area of a skin surface in which a stratum corneum is peeled by the stratum corneum peeling system.

Therefore, the microneedles need not to penetrate the stratum corneum. The thickness of the skin becomes thinner based on the thickness of the stratum corneum that is peeled off the skin. As a result, all or almost all of the microneedles result in reaching a live cell layer (epidermis below the prickle cell layer) of the skin, thereby effectively administering the medical fluid into the live cell layer of the skin or into the blood.

In addition, the peeling process of the stratum corneum causes little pain, and the burden of a patient is extremely light even the microneedles are inserted in the skin. Further, Langerhans cells will be activated by the peeling.

The stratum corneum is constantly peeled off in a constant cycle to be replaced by a new stratum corneum. Therefore, it is easy to peel off this portion artificially, and the peeling does not cause pain. Therefore, the burden of a patient is extremely light.

Further, the microneedles are positioned on an area of the surface of the skin, where the stratum corneum is peeled off by the stratum corneum peeling system, so that it becomes possible to certainly administer the medical fluid in the skin surface from which the stratum corneum is removed.

According to the present invention, the device body includes: a cylinder body in which a syringe filled with the medical fluid is movably inserted; and the needle form body attached to a tip portion of the syringe, so that the medical fluid can be provided from the syringe, without using an injection needle, to the needle form body and be administered in the skin.

According to the present invention, a skin enlarging section for enlarging the skin when the cylinder body is pressed against the skin, is provided at a lower end opening of the cylinder body, so that the medical fluid can be provided from the syringe, without using an injection needle, to the needle form body and be administered in the skin. In addition, without using a special tool, the skin is enlarged so that the skin surface can be enlarged thinly to effectively administer the medical fluid.

According to the present invention, the device body includes: a base sheet; a capsule-shaped medical fluid filling section provided on one surface of the base sheet; and the needle form body provided on the other surface of the base sheet; and a fragile portion is formed in the base sheet, the fragile portion being breakable by a pressure created by pressing the medical fluid filling section. Therefore, the device body is pressed against the skin after the stratum corneum peeling system is removed, so that the medical fluid filling section is pressed and the base sheet is broken. As a result, the medical fluid leaking therefrom can be delivered to the needle form body and administered in the skin.

According to the present invention, the connection section includes a supporting sheet provided on the side of the needle form body of the device body; the supporting sheet includes an opening formed therein, the opening corresponding to the needle form body; and the stratum corneum peeling system is connected to the device body with the supporting sheet interposed therebetween. Therefore, a press piece to be pressed by finger is provided at a part of the supporting sheet or an adhesive to be adhered to the skin is provided at a part of the supporting sheet, so that the needle form body can be positioned by pressing it by finger or adhering it by the adhesive.

According to the present invention, the connection section further includes a tearing sheet provided between the supporting sheet and the stratum corneum peeling system, and the tearing sheet can be torn along the skin surface so that it becomes possible to simultaneously tear the tearing sheet and the stratum corneum peeling system. Therefore, when the tearing sheet is pulled after the stratum corneum peeling system is pressed against the skin, the stratum corneum peeling system is peeled off the skin and the stratum corneum is removed. It becomes possible to press the needle form body through the opening of the remaining supporting sheet against the skin, off which the stratum corneum is peeled, to insert the microneedles into the skin, so that the medical fluid from the medical fluid filling section of the device body can be administered through the fluid passage hole and from the microneedles of the needle form body into the skin. Therefore, the tearing sheet is torn to simultaneously perform the removal of the stratum corneum and the removal of the supporting sheet. As a result, after the removal of the stratum corneum, the medical fluid can be immediately administered to perform the prompt process. Further, the needles and the needle form body are covered by the supporting sheet until immediately before the administration, so that asepsis of the needles can be maintained until immediately before the administration.

According to the present invention, the tearing sheet is constituted of: a first sheet positioned in the middle; and second and third sheets respectively positioned on both sides of the first sheet; the first sheet is adhered to end portions of the other two sheets, the end portions being folded; and the second sheet is connected to the supporting sheet and the third sheet is connected to a substrate sheet of the stratum corneum peeling system. By only pulling out the other end portion of the tearing sheet, the stratum corneum peeling system is removed from the skin, and simultaneously, the supporting sheet can be applied to the skin, thereby promptly administering the medical fluid.

According to the present invention, a pressure sensitive adhesive layer is provided at least at a part of a side contacting the skin of the supporting sheet, so that the positioning of the needle form body can be performed easily and certainly.

According to the present invention, the stratum corneum peeling system includes: a substrate sheet; and a pressure sensitive adhesive layer laminated on the substrate sheet, and the stratum corneum on the skin surface can be peeled by the pressure sensitive adhesive layer, so that the configuration of the stratum corneum peeling system becomes simple.

According to the present invention, the positioning sheet includes an opening, the device body is connected to one end portion of the positioning sheet such that the device body can be folded and piled on the positioning sheet, and the stratum corneum peeling system is connected to the other end portion of the positioning sheet such that the stratum corneum peeling system can be folded and piled on the positioning sheet, and the microneedles of the needle form body are configured to be positioned at the opening when the device body is folded and piled on the positioning sheet, and the pressure sensitive adhesive layer of the stratum corneum peeling system is configured to be positioned at the opening when the stratum corneum peeling system is folded and piled on the positioning sheet. Therefore, after the opening of the positioning sheet is applied to a predetermined position of the skin, and after the stratum corneum peeling system is folded to be pressed against the skin inside the opening to peel off the stratum corneum, the device body is folded and pressed against the skin inside the opening, and further, the medical fluid filling section is pressed to break the base sheet. As a result, the medical fluid leaking therefrom can be delivered to the needle form body and administered in the skin.

According to the present invention, the positioning sheet is formed in a quadrilateral shape in a planar view, the device body is connected to one side of the positioning sheet, and the stratum corneum peeling system is connected to the other side of the positioning sheet, so that the device body certainly contacts the skin, off which the stratum corneum is peeled. Further, the administering operation is simple.

According to the present invention, the positioning sheet is formed in a quadrilateral shape in a planar view, the device body is connected to one side of the positioning sheet, and the stratum corneum peeling system is connected to each of the other three sides of the positioning sheet, so that the stratum corneum is certainly peeled off the skin by the three stratum corneum peeling systems.

According to the present invention, the pressure sensitive adhesive layer is provided at least at a part of a side contacting the skin of the positioning sheet, so that the positioning of the needle form body is performed easily and certainly.

As described above, there is no need for the penetration of the stratum corneum, which is conventionally the largest problem for the microneedles. As a result, the height of the microneedles can be low; and even when the diameter of the microneedles is formed thin, a sufficient strength can be maintained. As a result, it becomes possible to reduce a time until the effect of medicines, such as insulin and anesthetic, appears and a time until the effect of a physiological active substance, such as DNA, RNA, protein and peptide, appears.

In the case where the surface provided with the microneedle assembling section is formed in a convex surface (with the radius of curvature of R 10 to 100 mm, and preferably 30 to 40 mm), when the microneedle assembling section is pressed against the skin of a human, all or a part of the microneedles enter the skin uniformly without unevenness. This provides a suitable efficiency. Furthermore, one or more fluid passage holes are penetratingly formed from the back surface to the front surface or the convex surface. Therefore, when the medical fluid is provided from the back surface side of the microneedle assembling section, the medical fluid moves to the front surface side, and flows along the convex surface or flows out from the midway of the convex surface, and moves to the base portion of the microneedles. Therefore, the medical fluid can be administered along the microneedles to the live cell layer (epidermis below the prickle cell layer) of the cuticle.

### BRIEF DESCRIPTION OF DRAWINGS

FIG. 1 is a perspective view of a transdermal administration device according to the present invention.

FIG. 2 is a cross sectional view of the transdermal administration device.

FIGS. 3(a) and 3(b) are respectively a side view of a needle form body and an enlarged view of a plane of the needle form body.

FIG. 4 is an enlarged cross sectional view of an essential part of the needle form body.

FIG. 5 is an enlarged cross sectional view of a microneedle.

FIGS. 6(a), 6(b) and 6(c) are cross sectional views illustrating forms of connecting modes of a tearing sheet and a stratum corneum peeling system.

FIG. 7 is a cross sectional view illustrating the transdermal administration device described above during its use.

FIG. 8 is a cross sectional view illustrating the transdermal administration device described above during its use.

FIG. 9 is a perspective view of an essential part of the transdermal administration device when another tearing sheet is used.

FIG. 10 is a perspective view illustrating the transdermal administration device described above during its use.

FIG. 11 is a perspective view of another form of the transdermal administration device.

FIG. 12 is a cross sectional view of the transdermal administration device.

FIG. 13 is a cross sectional view of the transdermal administration device described above during the administering of a medical fluid.

FIG. 14 is a plan view of still another form of the transdermal administration device.

FIG. 15 is a cross sectional view of the transdermal administration device described above.

FIGS. 16(a) and 16(b) are cross sectional views illustrating another connecting mode of the tearing sheet.

FIGS. 17(a) and 17(b) are plan views illustrating another connecting mode of the tearing sheet.

FIGS. 18(a) and 18(b) are plan views illustrating another connecting mode of the tearing sheet and an opening.

FIG. 19 is an enlarged cross sectional view illustrating various modes of a fluid passage hole provided in the microneedle.

FIG. 20 is a plan view of still another form of the transdermal administration device.

FIG. 21 is a cross sectional view of the transdermal administration device illustrated in FIG. 20.

FIG. 22 is a view of the transdermal administration device illustrated in FIG. 20, explaining its function.

FIG. 23 is a view of the transdermal administration device illustrated in FIG. 20, explaining its function.

1 microneedle
2 needle form body
4 fluid passage hole
5 device body
6 stratum corneum peeling system
22 supporting sheet
24 tearing sheet
26 opening
30 skin
38 connection section
A transdermal administration device

### DESCRIPTION OF EMBODIMENTS

Hereinafter, embodiments of the present invention will be described in detail with reference to the accompanying drawings.

### Embodiment 1

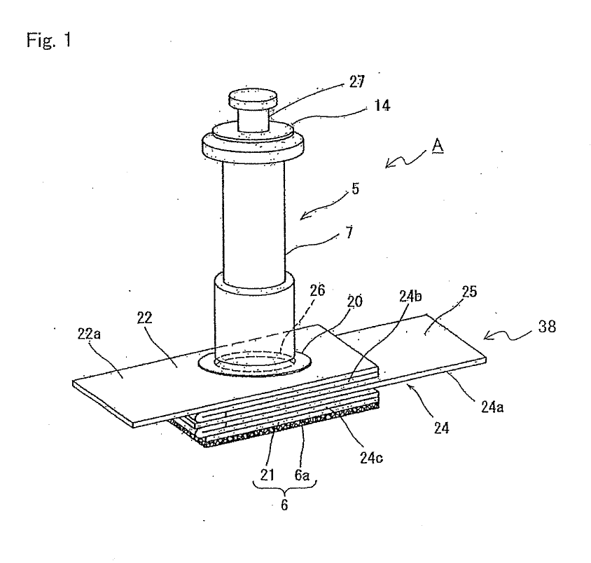

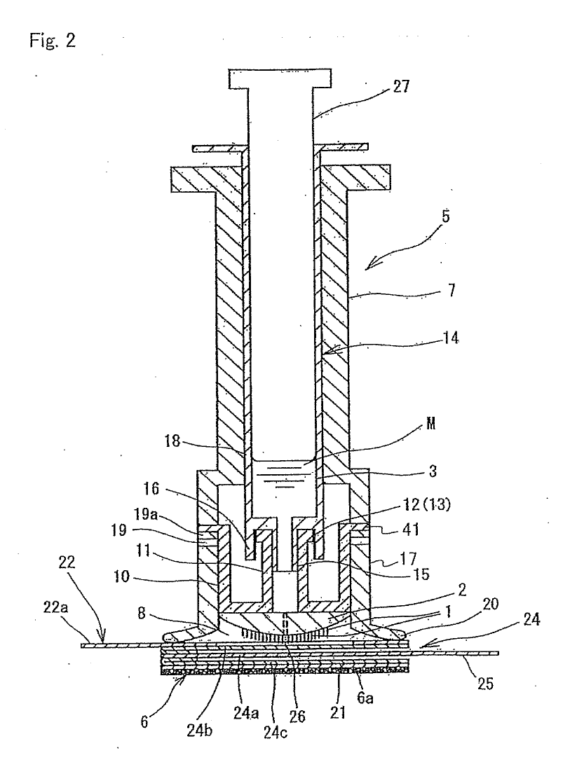

FIGS. 1 and 2 are respectively a perspective view and a longitudinal cross sectional view of a transdermal administration device A according to the present invention.

The transdermal administration device A includes: a device body 5; a stratum corneum peeling system 6; and a connection section 38 for connecting the device body 5 and the stratum corneum peeling system 6.

The device body 5 includes: a needle form body 2 including a plurality of microneedles 1 formed on a surface thereof; and a fluid passage hole 4 through a front surface to a back surface thereof; and a medical fluid filling section 3 for providing the medical fluid to the back surface of the needle form body 2.

The stratum corneum peeling system 6 includes: a substrate sheet 21; and a pressure sensitive adhesive layer 6a laminated on the substrate sheet 21, where the pressure sensitive adhesive layer 6a may peel a stratum corneum 30a off a surface of a skin.

The stratum corneum peeling system 6 is connected to the device body 5 by the connection section 38 such that the microneedles 1 of the needle form body 2 of the device body 5 are positioned on an area of the surface of the skin, where the stratum corneum 30a is peeled off by the pressure sensitive adhesive layer 6a of the stratum corneum peeling system 6.

Specifically, the transdermal administration device A includes: the device body 5 having the needle form body 2 including the plurality of microneedles 1 on the surface and the fluid passage hole 4 through the front surface to the back surface of the needle form body 2; and the medical fluid filling section 3 for providing a medical fluid M to the back surface of the needle form body 2; and the stratum corneum peeling system 6 provided with the pressure sensitive adhesive layer 6a for removing the stratum corneum 30a on the skin surface on the substrate sheet 21, and the device body 5 and the stratum corneum peeling system 6 are connected to each other through a supporting sheet 22 and a tearing sheet 24, which function as the connection section 38.

Note that the connecting includes, other than adhesive, a gentle connection by pressure sensitive adhesive.

The detail will be described hereinafter.

(Needle Form Body of the Device Body)

The device body 5 is constituted of a cylinder body 7, which includes the needle form body 2 and the medical fluid filling section 3.

As illustrated in FIGS. 2 to 4, the needle form body 2 described above has a surface, which is formed in a convex shape. The needle form body 2 is provided with a microneedle assembling section 8, which is a disk-plate or rectangle-plate member of 10 to 50 mm (preferably 20 to 40 mm) in diameter or of one side, and in which the plurality of microneedles 1 are assembled and positioned at a top section of the front surface. The microneedle assembling section 8 is a convex surface with the radius of curvature of R 10 to 100 mm (preferably 10 to 40 mm, and still preferably 30 to 40 mm).

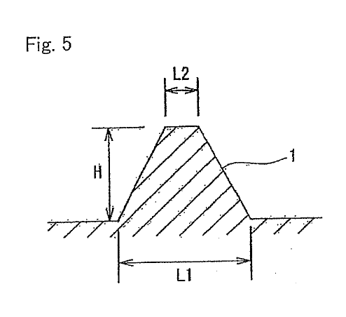

As illustrated in FIG. 5, the microneedles 1 are formed in a truncated cone and are formed such that a base diameter L1 is φ10 to 1000 μm (preferably 50 to 200 μm), a tip diameter L2 of the needle is 10 to 100 μm, and the length (height) H is 10 to 1000 μm (preferably 50 to 100 μm).

The surface of the needle form body 2 is provided with the microneedle assembling section 8, in which the plurality of microneedles 1 with the height of 10 to 1000 μm (preferably 50 to 100 μm) are assembled and positioned.

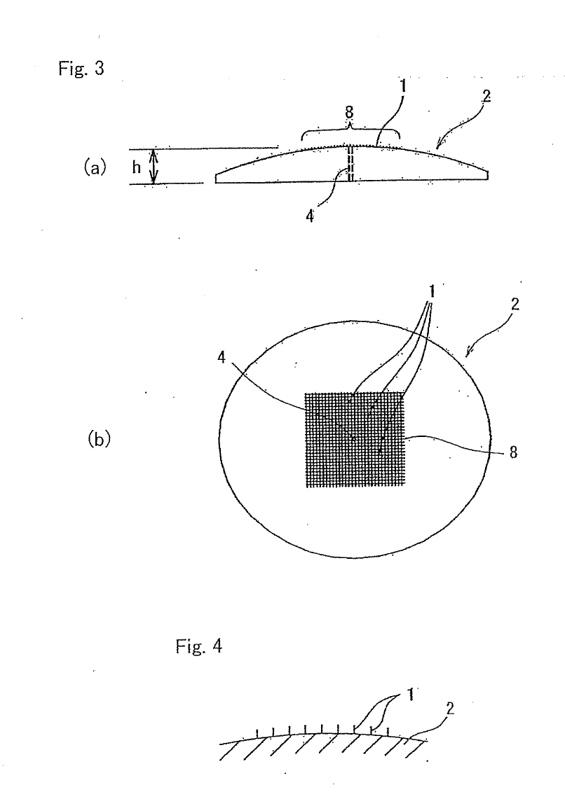

Furthermore, as illustrated in FIGS. 2 and 3, the microneedle assembling section 8 described above is provided with 1 to 1000 (preferably 100 to 650) of the microneedles 1 at 0.1 to 20 mm (preferably 0.5 to 1 mm) pitch.

Furthermore, at a center portion of the needle form body 2, one or more of the fluid passage holes 4 are penetratingly formed from the back surface to the front surface or the convex surface. The diameter of the opening is 5 mm or less, which may be a size that permits a medical fluid from the above, not to drip below, but to ooze through the opening. The position of the fluid passage hole 4 is not limited to the center portion. The fluid passage hole 4 may be provided in the periphery of the microneedle assembling section 8.

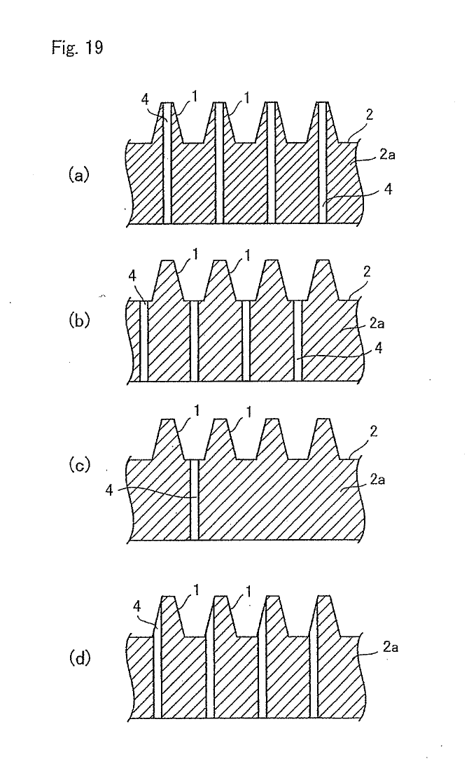

Furthermore, as illustrated in FIG. 19(a), the fluid passage hole 4 may be formed in such a manner to pass through the microneedle 1; or as illustrated in FIG. 19(b), the fluid passage hole 4 may be formed at a position where the fluid passage hole 4 passes through, not the microneedle 1, but only a body 2a of the needle form body 2. When the fluid passage hole 4 passes through the microneedle 1, it may pass through the tip portion of the microneedle 1. Alternatively, as illustrated in FIG. 19(d), the fluid passage hole 4 may be formed at a position offset from the tip portion of the microneedle 1. That is, the fluid passage hole 4 may be formed in such a manner that the fluid passage hole 4 passes through a slanting surface of the microneedle 1. As illustrated in FIG. 19(c), one fluid passage hole 4 may be formed, or two or more of the fluid passage holes 4 may be formed.

The microneedle 1 and the needle form body 2 may be manufactured as one integrated body by injection molding and low pressure casting, with a thermoplastic resin, such as polycarbonate, polypropylene, ABS resin, or polystyrene, or a thermosetting resin, a biocompatible resin or the like as a material.

When the microneedle 1 and the needle form body 2 are manufactured using a metal mold, it is necessary to open a large number of openings in a micron order size in the metal mold. Therefore, it is suitable to manufacture the metal mold by cutting work in micron bite, casting work with a punching tool, discharging work, etching work, electroforming work, or the like.

It is preferable for the diameter of the fluid passage hole 4, which is formed in the microneedle 1, to be φ 1 to 100 μm, and still preferably, to be φ 40 to 80 μm. When the diameter of the fluid passage hole 4 is small (e.g., φ 50 μm or less), the fluid passage hole 4 can be formed by a laser, a metal mold or drilling. When the fluid passage hole 4 is large (e.g., φ 50 μm or more), the fluid passage hole 4 can be formed by a metal mold, a laser or drilling.

In addition, the microneedle 1 may be formed of an inorganic material, such as silica, and a metal, such as stainless steel. In such a case, the microneedle 1 may be formed by a publicly known etching, pressing, insert molding, adhering and other methods.

(Device Body)

Next, the needle form body 2 is used by attaching to the transdermal administration device A.

The transdermal administration device A is configured such that a syringe 14 is inserted from above the cylinder body 7 and is screwed and fixed to a pedestal 10, the microneedle assembling section 8 is impregnated with a medical fluid, and subsequently, the syringe 14 is pumped so that the microneedles 1 of the needle form body 2, which is deposited at a deposition section to the pedestal 10, penetrate and pulled out the skin to provide the medical fluid described above for a live cell layer (epidermis below the prickle cell layer) of the cuticle.

That is the needle form body 2 is fixed to the pedestal 10, which is movably provided below the interior side of the cylinder body 7. The pedestal 10 is formed in a short cylinder shape having a cylinder portion 11 in the middle, and a male screw 12 is formed at exterior side of the tip of the cylinder portion 11. The male screw 12 is formed in a size screwable with a female screw 13 on the interior side of an outer circumference edge 16 formed on the outer circumference of an injection needle attaching section 15 at the tip of the syringe 14.

The needle form body 2 is welded and fixed on a lower surface of the pedestal 10. Furthermore, the needle form body 2 attached to the pedestal described above engages with the cylinder body 7.

Furthermore, an opening 19 is formed in the cylinder body 7, in which a coupling piece 19a is provided. A coupling section 41, which is a stick shape or a round shape, is protrudingly formed on the exterior side of the upper part of the pedestal 10. The coupling section 41 is coupled with the coupling piece 19a from the above. As a result, with the elasticity of the coupling piece 19a, the needle form body 2 attached to the pedestal is biased upwardly and is movable vertically within a predetermined range.

In the cylinder body 7, a housing section 17 is formed for housing the needle form body 2 attached to the pedestal in such a manner that the needle form body 2 is movable vertically. The upper part of the housing section 17 is formed in a size in which a cylinder 18 of the syringe 18 is insertable. In an opening of the lower end of the cylinder body 7, a circular edge 20 is formed as a skin enlarging section for enlarging the skin when the cylinder body 7 is pressed to a skin 30. The cylinder 18 and a piston 27 of the syringe 14 constitute the medical fluid filling section 3.

The device body 5 on the side of the needle form body 2 is covered with the supporting sheet 22, which is formed of a synthetic resin, a cloth material or the like, and the supporting sheet 22 is adhered and fixed to the lower end of the circular edge 20 of the cylinder body 7 described above. In the supporting sheet 22, an opening 26 is formed at a position corresponding to the needle form body 2 described above. An end portion 22a of the supporting sheet 22 extends out of the end portion of the stratum corneum peeling system 6.

Hereinafter, a further detail will be described.

The device body 5 includes: the cylinder body 7, in which the syringe 14 filled with a medical fluid is movably inserted; and the needle form body 2, which is attached to the tip portion of the syringe 14.

A flange is formed at the upper end portion of the cylinder body 7, and the housing section 17 with a slightly larger diameter is formed at the lower portion of the cylinder body 7. Further, the skin enlarging section 20 is integratedly provided in a slanting manner at the lower end portion of the housing section 17.

The needle form body 2 is connected to the tip of the syringe 14 with the pedestal 10 interposed therebetween. The needle form body 2 includes: a needle form body main body 2a, which is substantially a lens shape with a convex surface; and the plurality of microneedles 1 positioned at least at the center portion of the surface of the needle form body main body 2a. The microneedles 1 may be positioned on the entire surface of the needle form body main body 2a except the peripheral edge thereof.

In the center portion of the needle form body main body 2a, one or more fluid passage holes 4 are penetratingly formed from the back surface side to the front surface side of the main body.

The pedestal 10 is formed in a substantially cylinder shape, and includes: a tube section 11; an external tube; a circular connecting body for connecting the tube section 11 and the external tube; and a coupling section 41 extended outwardly from the external tube. A communicative connection hole is formed in the tube section 11. The needle form body 2 is fixed to the lower surface of the connecting body, and the fluid passage hole 4 formed in the needle form body 2 and the communicative connection hole are successively connected.

As illustrated in FIG. 2, the male screw 12 is formed at the end portion of the tube section 11, and the female screw 13 formed at the tip of the syringe 14 is screwed to the male screw 12 as described above.

In the housing section 17 formed at the tip portion of the syringe 14, each of the openings 19 is formed at a position such that the openings face with the other. The coupling piece 19a is extended in the horizontal direction from a part of the periphery of the opening 19. Further, the coupling section 41 of the pedestal 10 is inserted into the opening 19 in such a manner to couple with the coupling piece 19a. Therefore, since the coupling piece 19a may elastically change its form in the vertical direction, the pedestal 10 is movable in the vertical direction within the housing section 17.

When the lower portion of the cylinder body 7 is placed on the skin surface and the syringe 14 is pressed against the skin, the pedestal 10 and needle form body 2 provided at the lower end are moved downwardly and the microneedles 1 enter the skin. When the pressure to the syringe 14 is released, the syringe 14 is pushed upwardly by the restoring force of the coupling piece 19a and the microneedles 1 are pulled out of the skin. By repeating this pumping action, the microneedles 1 penetrate and are pulled out of the skin.

(Stratum Corneum Peeling System)

A place for administering a medicine is the skin. The stratum corneum 30a of the skin at the site for the administering is removed in advance as much as possible by the stratum corneum peeling system 6. The stratum corneum 30a is constantly peeled off in a natural state to be grime, and a new stratum corneum is produced. Therefore, it is easy to peel off this portion artificially, and the peeling does not cause pain. The thickness of the stratum corneum 30a is 10 to 20 μm, and the thickness varies depending on the age, sex and the like. The same applies to the elasticity of the stratum corneum 30a.

As the stratum corneum peeling system 6, it is desirable to use a peeling sheet provided with the pressure sensitive adhesive layer 6a for removing the stratum corneum 30a of the skin surface, on the substrate sheet 21.

The peeling sheet peels off the stratum corneum 30a of the surface by adhering to the skin, and therefore, it is a member in which the pressure sensitive adhesive layer 6a is laminated on the lower surface of the substrate sheet 21. For example, what can be used as the substrate sheet 21 includes: a plastic film constituted of a polyester resin of polyethylene terephthalate and the like, a polyolefin resin constituted of polyethylene, polypropylene, ethylene/methacrylic acid copolymer, ethylene/methyl methacrylic acid copolymer or the like, or a thermoplastic elastomer resin such as polyurethane resin and polystyrene; a textile constituted of various synthetic fibers and/or natural fibers; fabric and nonwoven fabric; paper such as high quality paper, glassine paper, coated paper; and a laminated body thereof. The thickness of the substrate sheet 21 is preferably, on average, 10 to 800 μm.

The pressure sensitive adhesive layer 6a has a function to remove an old stratum corneum 30a from the skin surface by removing itself from the surface of the living body after being adhered thereto. Any pressure sensitive adhesive can be used as long as the stratum corneum 30a is removed from the skin surface by its adhesion, including, for example, a rubber pressure sensitive adhesive, an acrylic pressure sensitive adhesive, and a silicone pressure sensitive adhesive. Furthermore, the adhesion of the pressure sensitive adhesive layer 6a (mode: 180° peeling, peeling speed: 300 mm/min, applying duration: five minutes), which is measured conforming to JIS 20237 five minutes after the applying to bakelite, is preferably 2 to 20 N/25 mm. Except for the peeling sheet described above, different kinds of stratum corneum peeling systems 6, which can certainly remove a corneum, can be used.

(Connecting Section)

The stratum corneum peeling system 6 described above is connected to the device body 5 by the connection section 38 so that the microneedles 1 of the needle form body 2 of the device body 5 is positioned in the area of the skin surface, where the stratum corneum is removed by the pressure sensitive adhesive layer 6a of the stratum corneum peeling system 6.

In the embodiment illustrated in FIG. 2, the tearing sheet 24, which functions as a connection section, is provided between the supporting sheet 22 and the peeling sheet 6 of the device body 5.

The tearing sheet 24 is made of a synthetic resin, a cloth material or the like. The tearing sheet 24 is tearably attached on the back surface of the supporting sheet 22 and is configured to be able to tear the stratum corneum peeling system 6 from one end to the other end.

As illustrated in FIG. 2, the tearing sheet 24 includes a first tearing sheet 24a, a second tearing sheet 24b and a third tearing sheet 24c. The end portions of the second tearing sheet 24b and third tearing sheet 24c are folded back, and the first tearing sheet 24a extends out of the other tearing sheets 24b and 24c as an extending portion 25. The extending portion 25 is configured to be pinched by hand from the outside to pull out the first tearing sheet 24a.

The second tearing sheet 24b is integratedly adhered to the back surface of the supporting sheet 22, and the third tearing sheet 24c is integratedly adhered to the stratum corneum peeling system 6.

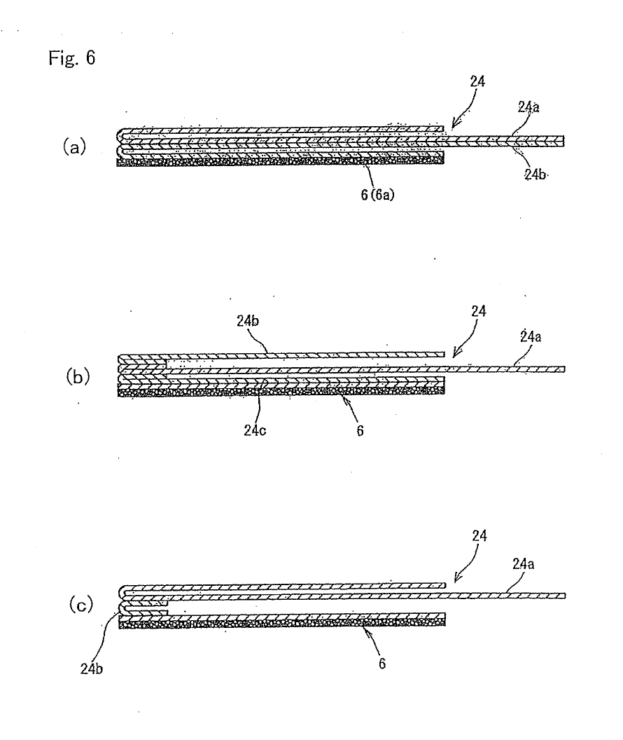

Note that the configuration of the tearing sheet 24 and the stratum corneum peeling system 6 is not limited to the above-described form. It is suitable as long as the tearing sheet 24 is configured to be tearable from the back surface of the supporting sheet 22 and to be able to tear the stratum corneum peeling system 6 from the skin. A part of the third tearing sheet 24c may also serve as the substrate sheet 21 of the stratum corneum peeling system 6. Other embodiments of the tearing sheet 24 are illustrated in FIG. 6.

For example, the tearing sheet 24 illustrated in FIG. 6(a) is constituted of the first tearing sheet 24a and the second tearing sheet 24b. The end portions of the first tearing sheet 24a and second tearing sheet 24b are respectively folded back to the opposite side. The folded portion of the first tearing sheet 24a is integratedly adhered to the back surface of the supporting sheet 22 described above, and the folded portion of the second tearing sheet 24b is integratedly adhered to the stratum corneum peeling system 6. In the above embodiment, a part of the tearing sheet 24 also serves as the substrate sheet 21 of the stratum corneum peeling system 6.

As illustrated in FIG. 6(b), the tearing sheet 24 may be constituted of three sheets 24a, 24b and 24c with each of the end portions folded. The respective folded portions of the three sheets 24a, 24b and 24c are adhered.

As illustrated in FIG. 6(c), it is also possible to fold one long sheet 24a, adhere one folded end of the short sheet 24b to the folded portion of the sheet 24a, and adhere the other end of the sheet 24b to the end portion of the stratum corneum peeling system 6.

The adhesion of the supporting sheet 22 and the tearing sheet 24 is performed by an adhesive. It is also possible to apply a pressure sensitive adhesive appropriately to the side contacting the skin of the supporting sheet 22. In such a case, it is further possible to provide a release agent on the side of the tearing sheet contacting the supporting sheet, so that the pressure sensitive adhesive surface is exposed at the tearing to securely fix the supporting sheet at a target position of the skin.

(Method for Administering)

To use the transdermal administration device A, first, the syringe 14 is removed from the cylinder body 7, the piston 27 is pulled out of the syringe 14, and the medical fluid M is poured and filled inside the cylinder (medical fluid filling section 3). Subsequently, the syringe 14 is turned up and inserted in the cylinder body 7, and the male screw 12 of the cylinder portion 11 of the pedestal 10 is screwed to the female screw 13 of the outer circumference edge 16 of the syringe 14, so that the injection needle attaching section 15 at the tip of the syringe 14 is attached to the pedestal 10. As a result, the attaching section 15 of the syringe 14 is communicatively connected to the cylinder portion 11 of the pedestal 10 (FIG. 2).

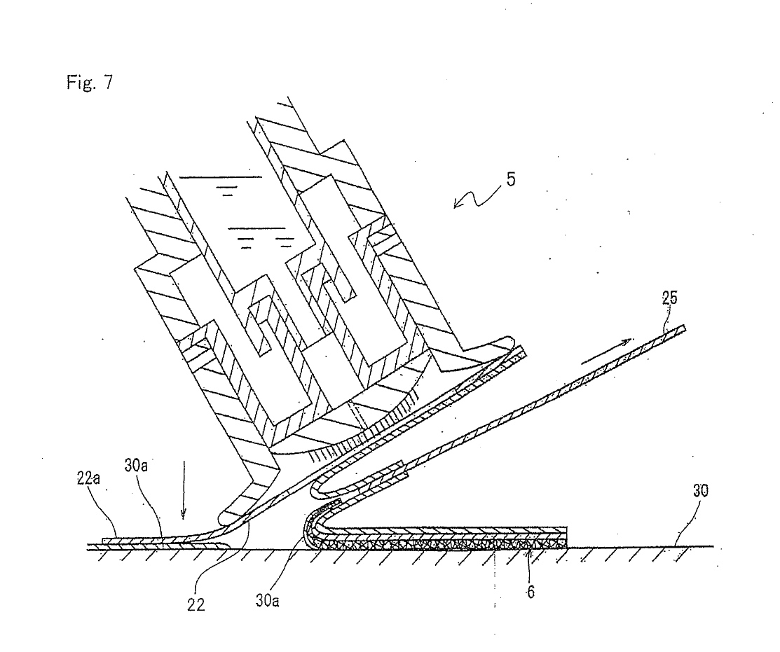

Next, as illustrated in FIG. 7, release paper (not shown) is peeled off the stratum corneum peeling system 6, and the pressure sensitive adhesive layer 6a of the stratum corneum peeling system 6 is adhered to the skin 30.

Next, the extending portion 22a of the supporting sheet 22 is pressed against the skin by hand (it is also possible to fix it to the skin by the pressure sensitive adhesive provided on the lower surface of the supporting sheet 22), and the extending portion 25 of the tearing sheet 24 is pinched and pulled out along the skin 30. As a result, the second tearing sheet 24b is removed from the supporting sheet 22, and the stratum corneum peeling system 6 is peeled off the skin 30, together with the third tearing sheet 240. At the same time, the stratum corneum 30a is adhered to the pressure sensitive adhesive layer 6a and is peeled off.

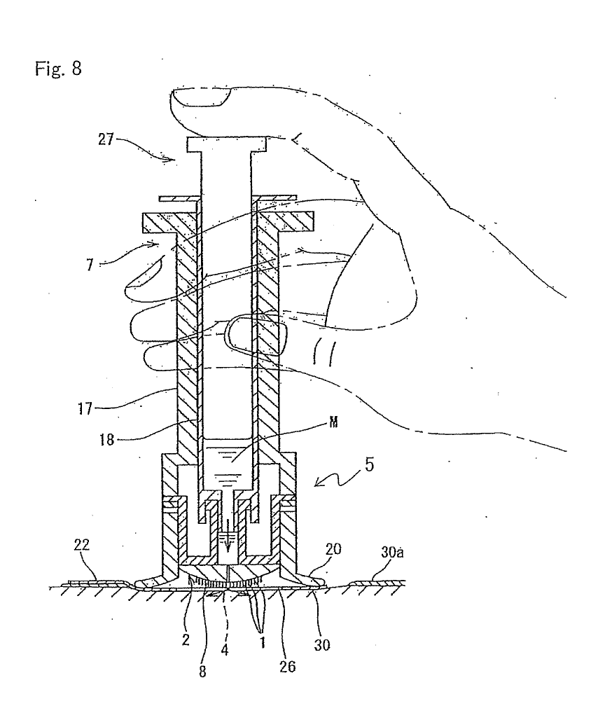

As illustrated in FIG. 8, the opening 26 of the supporting sheet 22 faces the portion of the skin 30, off which the stratum corneum 30a is peeled, after the stratum corneum peeling system 6 is removed, so that the needle form body 2 at the lower portion of the cylinder body 7 can contact the skin 30.

Next, the tip of the transdermal administration device A is placed at the portion of the skin 30, off which the stratum corneum 30a is peeled, and the piston 27 of the syringe 14 is pressed to ooze out the medical fluid M from the cylinder 18 via the fluid passage hole 4 to the microneedle assembling section 8 and the skin. Further, by pumping the piston 27 of the syringe 14, the medical fluid M is provided to the live cell layer of the cuticle (epidermis below the prickle cell layer) along the microneedles 1. Further, by the pumping, the circular edge 20 of the lower end opening of the cylinder body 7 is pressed against the skin 30, so that the skin 30 is enlarged and stretched out thinly. As a result, the microneedles 1 can enter the live cell layer of the cuticle (epidermis below the prickle cell layer) more securely to administer the medical fluid M. At the same time, the circular edge 20 and the edge of the opening 26 of the supporting sheet 22 are closely positioned to the skin 30, thereby preventing the medical fluid M from flowing out to an undesired area during the administering.

In addition, it is preferable to configure the needle form body 2 attached to the pedestal by an appropriate method such that the needle form body 2 attached to the pedestal is movable vertically relative to the cylinder body 7.

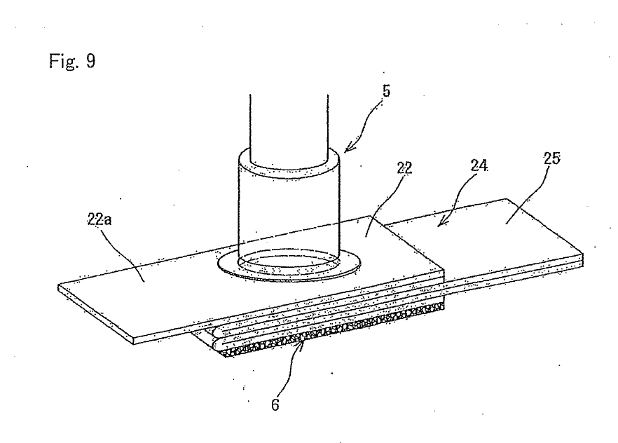

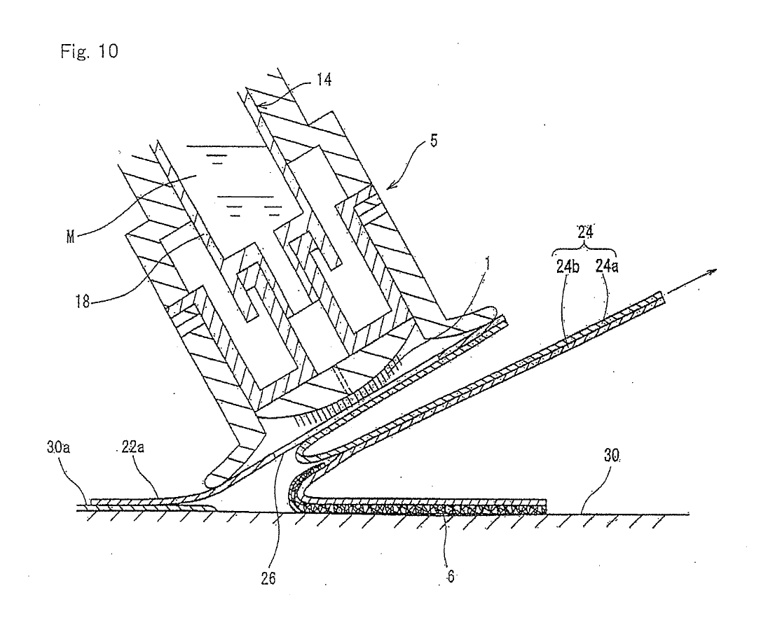

FIGS. 9 and 10 are perspective views of a part of the transdermal administration device adopted with the embodiment of FIG. 6(a) of the tearing sheet 24.

Also in this case, the extending portion 22a of the supporting sheet 22 is pressed against the skin by the finger and the tearing sheet 24 is pulled out along the skin 30, so that the first tearing sheet 24a is removed from the supporting sheet 22 and the stratum corneum peeling system 6 is peeled off the skin 30 with stratum corneum 30a, together with the second tearing sheet 24b. Subsequently, the opening 26 of the supporting sheet 22 faces the position where the stratum corneum 30a is peeled off, so that the medical fluid M can be provided from the syringe 14 along the microneedles 1 to the live cell layer of the cuticle, as similar to the previously mentioned case.

### Embodiment 2

Next, another embodiment of the transdermal administration device A will be described.

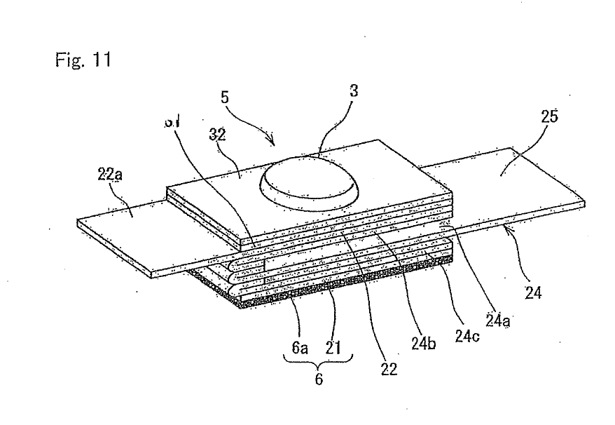

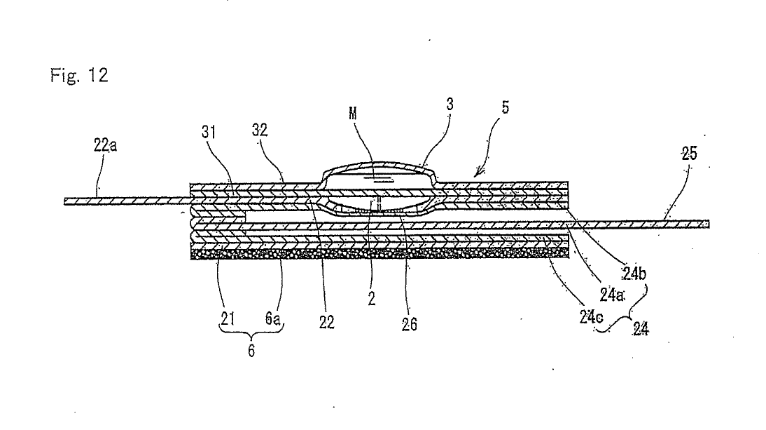

As illustrated in FIGS. 11 and 12, the device body 5 is configured by adhering a synthetic resin sheet 32 on a surface of an aluminum thin base sheet 31, forming a capsule-shaped medical fluid filling section 3 by the synthetic resin sheet 32, and a needle form body 2 is held on a back side of the base sheet 31 by a supporting sheet 22. By pressing the medical fluid filling section 3 from the above, a fragile portion formed in the base sheet 31 is broken and opened by the pressure, and a medical fluid in the medical fluid filling section 3 moves to the needle form body 2. An opening 26 is formed at a position in the supporting sheet 22 adhered on the back side of the needle form body 2, the position corresponding to the needle form body 2.

The stratum corneum peeling system 6 is connected to the back side of the device body 5 with the tearing sheet 24 interposed therebetween. The tearing sheet 24 and the stratum corneum peeling system 6 are configured the same as in FIG. 6(b). The tearing sheet 24 is provided between the supporting sheet 22 and the stratum corneum peeling system 6.

The stratum corneum peeling system 6 and the tearing sheet 24 may have a different configuration.

As similar to the previously described case, when the transdermal administration device A is used, release paper (not shown) is peeled off the stratum corneum peeling system 6, the extending portion 22a of the supporting sheet 22 is pressed by finger and the pressure sensitive adhesive layer 6a on the lower surface of the stratum corneum peeling system 6 is adhered to the skin 30.

Next, the tearing sheet 24 is pulled out along the skin 30. As a result, the stratum corneum peeling system 6 is peeled from the skin 30 with the stratum corneum 30a adhered on the pressure sensitive adhesive layer 6a. After the stratum corneum peeling system 6 is removed, the needle form body 2 contacts the position where the stratum corneum 30a is peeled off.

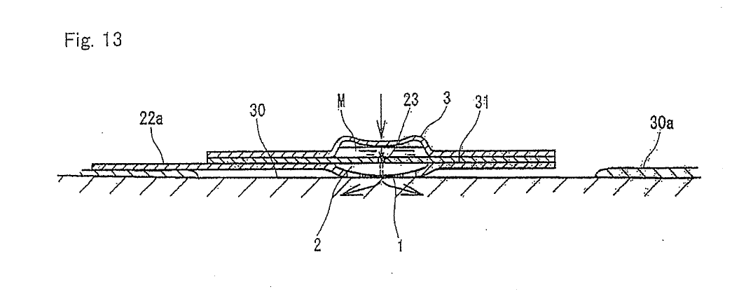

Next, as illustrated in FIG. 13, when the medical fluid filling section 3 is pressed from the above, the medical fluid M moves from the opening 23, which is formed when the base sheet 31 is broken, to the needle form body 2. Further, the medical fluid M passes through the fluid passage hole 4 and oozes to the microneedle assembling section 8 and the skin 30. By pressing the medical fluid filling section 3 as described above, the medical fluid M can be administered to the live cell layer of the cuticle along the microneedles 1. According to the above configuration, it is possible to administer the medical fluid promptly.

Note that the extending portion for pressing the device body 5 by finger, may be provided, not at the supporting sheet 22, but at the base sheet 31.

### Embodiment 3

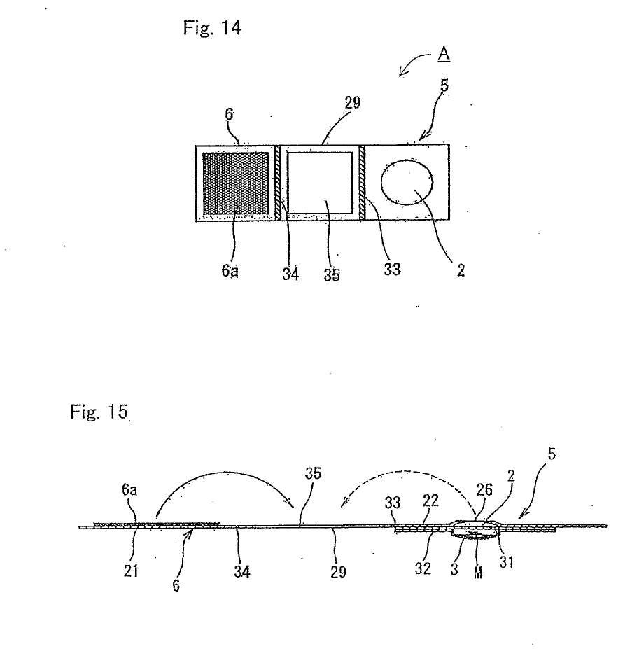

Next, still another embodiment of the transdermal administration device A will be described with reference to FIGS. 14 and 15.

The transdermal administration device A herein is configured by arranging and connecting a device body 5 and a stratum corneum peeling system 6 with a positioning sheet 29 functioning as a connection section interposed therebetween.

The device body 5 is configured by adhering a synthetic resin sheet 32 on a surface of an aluminum thin base sheet 31, swelling out and forming a capsule-shaped medical fluid filling section 3 by the synthetic resin sheet 32, and a needle form body 2 is applied on a back side of the base sheet 31 by a supporting sheet. By pressing the medical fluid filling section 3 from the above, a fragile portion formed in the base sheet 31 is broken and opened by the pressure, and a medical fluid in the medical fluid filling section 3 moves to the needle form body 2.

A fluid passage hole 4 is formed in the middle of the needle form body 2. Furthermore, an opening 26 is formed at a position in the supporting sheet 22 adhered on the back side of the needle form body 2, the position corresponding to the needle form body 2. The opening 26 is usually covered by release paper.

A positioning sheet 29 is foldably connected to one end of the device body 5. A connecting section 33, which is in between the device body 5 and the positioning sheet 29, is formed of a flexible member so that the device body 5 and the positioning sheet 29 can be folded. The positioning sheet 29 includes formed therein an opening 35 for positioning.

A portion contacting the skin 30 of the positioning sheet 29 is provided with a pressure sensitive adhesive layer, so that the positioning sheet 29 can be securely fixed to a target position of the skin. The stratum corneum peeling system 6 is foldably connected to an end portion of the positioning sheet 29 opposite from the device body 5. Similar to the previously described stratum corneum peeling system, the stratum corneum peeling system 6 is formed by laminating the pressure sensitive adhesive layer 6a on the lower surface of the substrate sheet 21. A connecting section 34, which is in between the positioning sheet 29 and the stratum corneum peeling system 6, is also formed of a flexible member.

When either the device body 5 or the stratum corneum peeling system 6 is folded to the positioning sheet 29 side, it faces the opening 35 of the positioning sheet 29.

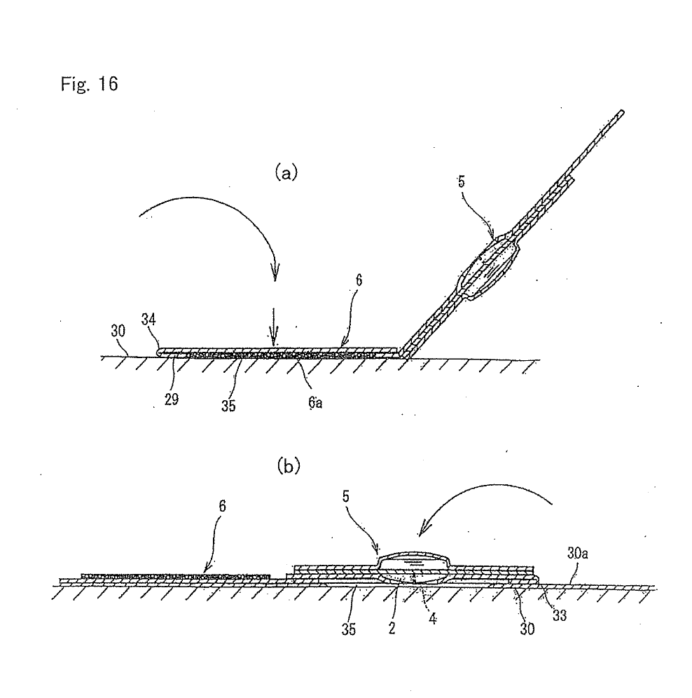

As illustrated in FIG. 16(a), when the transdermal administration device A described above is used, the opening 35 of the positioning sheet 29 is placed at a predetermined position of the skin 30, the release paper is peeled off the device body 5 and the stratum corneum peeling system 6, the stratum corneum peeling system 6 is pivoted 180 degrees relative to the connecting section 34, and the pressure sensitive adhesive layer 6a is applied to the skin 30. Next, the stratum corneum peeling system 6 is folded back to the original state, so that the pressure sensitive adhesive layer 6a of the stratum corneum peeling system 6 is peeled off the skin 30, with the stratum corneum 30a adhered to the pressure sensitive adhesive layer 6a.

As illustrated in FIG. 16(b), after the stratum corneum peeling system 6 is peeled off, the device body 5 is pivoted with the connecting section 33 as the center to contact the needle form body 2 of the device body 5 to the surface of the skin 30 where the stratum corneum 30a is peeled off.

Subsequently, when the medical fluid filling section 3 is pressed from the above, the base sheet 31 is broken and the medical fluid is moved to the needle form body 2, and further passing through the fluid passage hole 4 and oozing to the microneedle assembling section 8 and the skin 30. It is also possible to press and release the medical fluid filling section 3. When the medical fluid filling section 3 is pressurized, the medical fluid spouts out from the tip of the microneedles 1 like a jet, and the medical fluid is administered to the live cell layer of the cuticle along the microneedles 1.

As described above, the opening 35 of the positioning sheet 29 is adjusted to the predetermined position of the skin and the medical fluid is administered in such a state from the device body 5, thereby administering the medical fluid to the target portion of the skin accurately.

### Embodiment 4

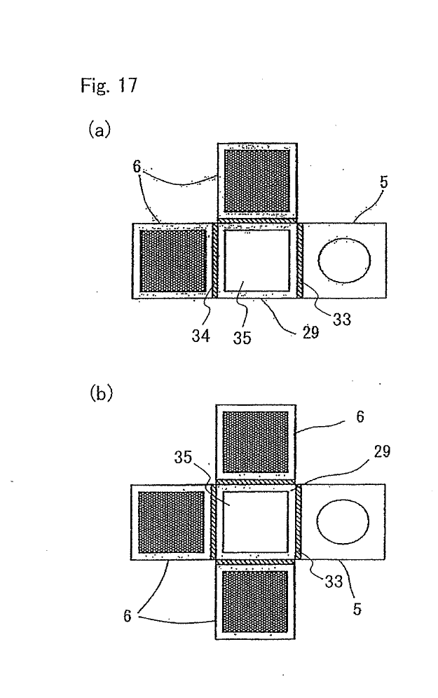

As illustrated in FIGS. 17(a) and 17(b), a plurality of stratum corneum peeling systems 6 may be connected to two or three sides of the positioning sheet 29.

In this embodiment, a stratum corneum 30a is peeled off by two or three sheets of a stratum corneum peeling systems 6, so that the stratum corneum 30a can be securely peeled. Furthermore, the adhesion per stratum corneum peeling system 6 can be reduced.

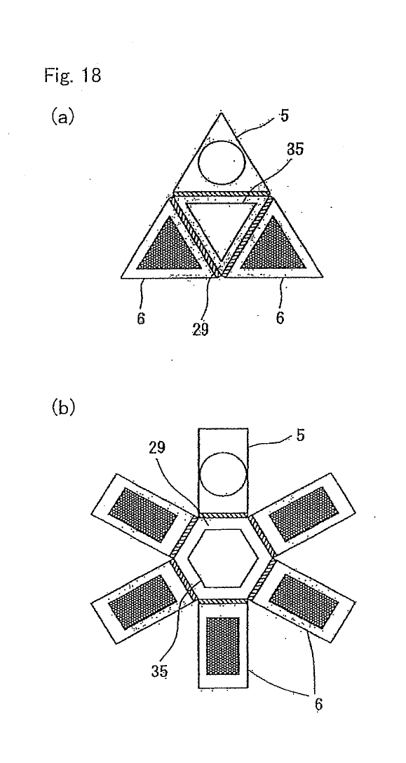

The shape of an opening 35 provided in a positioning sheet 29 is not limited to a square. For example, as illustrated in FIG. 18(a), the positioning sheet 29 and the opening 35 may be formed in a triangle; and as illustrated in FIG. 18(b), they may be formed in a hexagon. Of course, it is not required to connect the stratum corneum peeling systems 6 to all the sides of the positioning sheet 29.

According to the transdermal administration device A of each of the embodiments described above, a skin 30 becomes as thin as the thickness of the peeled stratum corneum 30a. Therefore, there is no need for the microneedles 1 to penetrate the stratum corneum 30a. By setting the height of the microneedles 1 to 10 to 1000 μm (preferably 50 to 100 μm), it has been confirmed that all or almost all the microneedles 1 reach the live cell layer (epidermis below the prickle cell layer) of the cuticle.

Furthermore, the microneedle assembling section 8 is provided with 1 to 1000 of the microneedles 1 at 0.1 to 20 mm (preferably 0.5 to 1 mm) pitch, so that the medical fluid can be provided effectively to the live cell layer (epidermis below the prickle cell layer) of the cuticle.

Furthermore, since it is possible to shorten the length of the microneedles 1, a sufficient strength can be maintained even when the base diameter is φ10 to 1000 μm (preferably 50 to 200 μm) and a tip diameter of the needle is 10 to 100 μm, thereby preventing the microneedles 1 from snapping or breaking in the body during the administering of the medical fluid, and avoiding the microneedles 1 from being left in the body before it happens.

Furthermore, the surface of the needle form body 2 is formed in a convex shape, so that, when the needle form body 2 is pressed against the skin 30, the microneedle assembling section 8 is concentratedly pressed hard and the pressure is not spread. Therefore, almost all the microneedles 1 are inserted into the skin 30, thereby effectively providing the medical fluid M in the skin 30.

Consequently, it becomes possible to reduce a time until the effect of medicines, such as insulin and anesthetic, appears and a time until the effect of a physiological active substance, such as DNA, RNA, protein and peptide, appears.

Furthermore, the peeling of the stratum corneum does not cause pain and the microneedles 1 do not reach the pain spot, so that the burden of a patient can be extremely light.

Except for a circular shape, the needle form body 2 may be in a shape of a square, an ellipse or the like. In addition, the surface of the microneedle assembling section 8, which is a surface contacting the skin 30, is not limited to a convex shape, but may be a planar shape. In addition, the shape of the microneedles 1 is not limited to the one described above.

Furthermore, the needle section is not limited to the circular cone shape, but may be a triangular pyramid, a quadrangular pyramid or the like. Furthermore, a groove for passing the administered medical fluid may be formed in the height direction. Furthermore, a through hole may be provided from the vicinity of the needle portion to the back surface of the needle form body 2.

### Embodiment 5

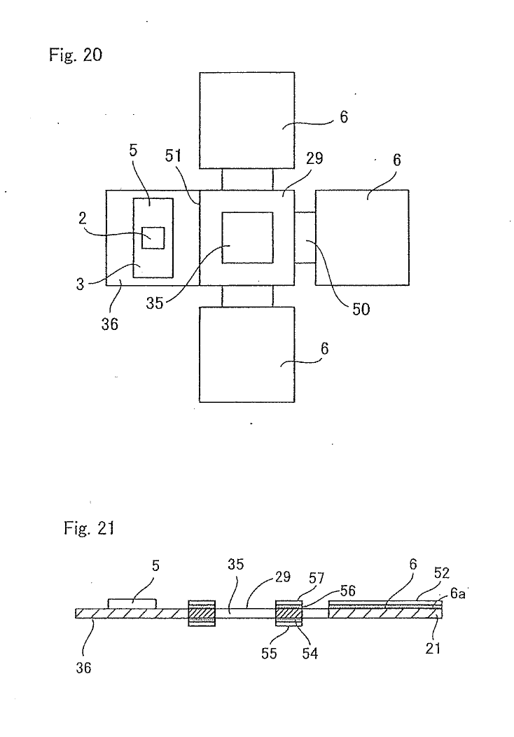

FIGS. 20 to 23 illustrate still another embodiment of the transdermal administration device.

The device includes: a quadrilateral positioning sheet 29 having a quadrilateral opening; three stratum corneum peeling systems 6 connected to three sides of the positioning sheet 29 with a folding sections 50 interposed therebetween; and a device body 5 connected to the rest of one side of the positioning sheet 29 with a folding section 51 interposed therebetween.

Each of the stratum corneum peeling systems 6 includes: a substrate sheet 21; and a pressure sensitive adhesive layer 6a laminated on the substrate sheet 21. The substrate sheet 21 is extended from the positioning sheet 29 integratedly. Normally, a liner 52 is peelably adhered to a pressure sensitive adhesive layer 6a. In addition, a pressure sensitive adhesive layer 54 and a liner 55 may be provided on a surface of the positioning sheet 29 contacting the skin. Furthermore, a pressure sensitive adhesive layer 56 with a liner 57 may be provided on a surface of the positioning sheet 29 that does not contact the skin.

The device body 5 is adhered to an extension piece 36 extended from the positioning sheet 29. The device body 5 may be applied by adhesive on the extension piece 36, or the device body 5 may be formed integratedly with the extension piece 36.

The device body 5 includes: a bag-form medical fluid filling section 3 for housing a medical fluid; and a needle form body 2 having a plurality of microneedles, the needle form body being attached to an external surface of the medical fluid filling section 3. On the external surface of the medical fluid filling section 3 on which the needle form body 2 is attached, a fragile portion (not shown) is formed, where the medical fluid moves by pressure from the medical fluid filling section 3 to the back surface side of the needle form body 2. The fragile portion may be formed of a slit or a pore of a thin portion of the medical fluid filling section 3.

When the device body 5 is folded and piled (or bent) on the positioning sheet 29, the microneedles of the needle form body 2 position at the opening 35. When the stratum corneum peeling systems 6 is folded and piled on the positioning sheet 29, the pressure sensitive adhesive layer 6a of the stratum corneum peeling systems 6 is configured to position at the opening 35.

The transdermal administration device in this embodiment is used as follows.

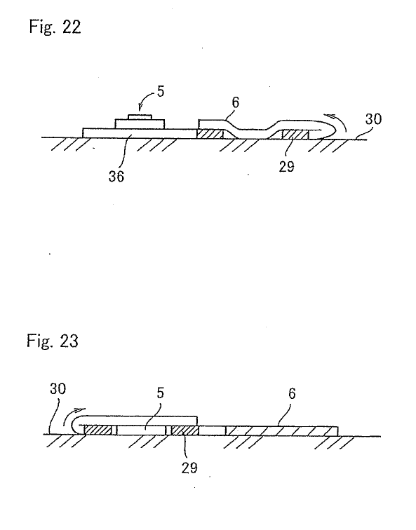

The positioning sheet 29 is positioned at a predetermined position of the skin, and the pressure sensitive adhesive layer 54, which is provided on the skin side surface of the positioning sheet 29, is applied to the skin as necessary. Subsequently, the stratum corneum peeling systems 6 is folded and piled on the opening 35 of the positioning sheet 29 to be applied on the skin (FIG. 22). When the stratum corneum peeling systems 6 is peeled off the skin, the stratum corneum is peeled while being adhered to the pressure sensitive adhesive layer 6a. As described above, this operation of applying the stratum corneum peeling systems 6 to the skin surface for peeling is successively repeated for three pieces of the stratum corneum peeling systems 6, and subsequently, the device body 5 is folded and piled on the positioning sheet 29 (FIG. 23). Herein, the pressure sensitive adhesive layer is provided on the opposite side of the skin of the extension piece 36 or positioning sheet 29, so that the extension piece 36 can be adhered on the positioning sheet 29. When the medical fluid filling section 3 of the device body 5 is pressed by finger or the like in this state, the fragile portion of the medical fluid filling section 3 is broken and the medical fluid flows from the medical fluid filling section 3 to the side of the needle form body 2. Subsequently, the medical fluid passes through the fluid passage hole and is administered to the surface of the microneedles and into the skin.

In Embodiment 5, the device body 5 is adhered on the extension piece 36 of the positioning sheet 29; however, the extension piece 36 may be a part of the medical fluid filling section 3 that constitutes the device body 5. That is, the extension piece 36 may be covered with a covering piece to configure the bag-form medical fluid filling section 3 and the needle form body 2 may be adhered to the extension piece 36 or the covering piece.

Hereinafter, the present invention will be specifically described with reference to examples; however, the present invention is not limited in anyway to the example.

### Example 1

In the form of FIG. 2, the needle form body 2 was φ20 mm in external diameter and 1 mm in thickness, and the polycarbonate microneedles 1 of a truncated cone were 100 μm in height, φ200 μm in base diameter and 20 μm in tip diameter. The needle form body 2 was formed by putting 121 microneedles 1 at the pitch of 1 mm.

At the tip side of the microneedles 1, the polyethylene supporting sheet 22 having the opening 26 was fixed at the tip portion of the cylinder body 7, and the peeling sheet with the configuration of FIG. 2 (formed with a polyester film of 50 μm in thickness by being appropriately bent and adhered) was adhered to the supporting sheet 22 to form the transdermal administration device.

As the pressure sensitive adhesive layer of the peeling sheet, an acrylic pressure sensitive adhesive (10 N/25 mm in adhesion) manufactured by Lintec Corporation was used.

Using the transdermal administration device, after the stratum corneum was removed by the peeling sheet, medical fluids containing 1 mg, 10 mg and 20 mg of histamine (manufactured by Sigma) per 1 g of hydrophilic ointment were transdermally administered to the skin. In each of the cases, hives were developed. Thus, it was verified that the needles of the microneedles 1 certainly passed through the stratum corneum and reached the live cell layer (epidermis below the prickle cell layer) of the skin.

### Example 2

In the form of FIG. 2, the needle form body 2 was φ20 mm in external diameter and had a convex shape (the thickness to the top of the convex surface was 4 mm and the R was 39 mm), and the polycarbonate microneedles 1 of a truncated cone were 100 μm in height, φ70 μm in base diameter and 10 μm in tip diameter. The needle form body 2 was formed by putting 121 microneedles 1 at the pitch of 1 mm.

At the tip side of the microneedles 1, the polyethylene supporting sheet having the opening was fixed at the tip portion of the cylinder body, and the peeling sheet with the configuration of FIG. 2 (formed with a polyester film of 50 μm in thickness by being appropriately bent and adhered) was adhered to the supporting sheet to form the transdermal administration device.

As the pressure sensitive adhesive layer of the peeling sheet, an acrylic pressure sensitive adhesive (10 N/25 mm in adhesion) manufactured by Lintec Corporation was used.

Using the transdermal administration device, the stratum corneum was removed by the peeling sheet. Medical fluids containing 1 mg, 10 mg and 20 mg of histamine (manufactured by Sigma) per 1 g of hydrophilic ointment were transdermally administered to the skin. In each of the cases, hives were developed. Thus, it was verified that the needles of the microneedles 1 certainly passed through the stratum corneum and reached the live cell layer (epidermis below the prickle cell layer) of the skin.

### Example 3

In the form of FIG. 2, the needle form body 2 was φ20 mm in external diameter and 1 mm in thickness, and the polycarbonate microneedles 1 of a truncated cone were 100 μm in height, φ0200 μm in base diameter and 20 μm in tip diameter. The needle form body 2 was formed by putting 121 microneedles 1 at the pitch of 1 mm.

At the tip side of the microneedles 1, the polyethylene supporting sheet having the opening was fixed at the tip portion of the cylinder body. The peeling sheet with the configuration of FIG. 2 (formed with a polyester film of 50 μm in thickness by being appropriately bent and adhered) was adhered to the supporting sheet to form the transdermal administration device.

As the pressure sensitive adhesive layer of the peeling sheet, an acrylic pressure sensitive adhesive (10 N/25 mm in adhesion) manufactured by Lintec Corporation was used.

Using the transdermal administration device, the stratum corneum was removed by the peeling sheet. Subsequently, 1 mg of a medical fluid, in which protein peroxidase (manufactured by Sigma) and ovalbumin (OVA) were dissolved in a physiological saline solution, was applied to the skin.

The microneedles 1 were pressed against the skin, in which the stratum corneum was removed by the peeling sheet, and the skin was observed after the elapse of 24 hours. As a result, it was verified that the protein peroxidase and OVA existed in the live cell layer (epidermis below the prickle cell layer) of the skin under the stratum corneum, and that pores were made by the microneedles penetrating the stratum corneum.

When the section of the skin was died with hematoxylin-eosin stain and a cut piece of the section was observed with an optical microscope, it was confirmed that the needles of the microneedles 1 reached the live cell layer (epidermis below the prickle cell layer) of the skin. When the skin was observed through serial sections, it was confirmed that holes were made by the needles of the microneedles 1.

### Example 4

In the form of FIG. 20, the device body 5 was formed of the polycarbonate medical fluid filling section 3, which was cast in a bag-form of 45 mm×25 mm; and the needle form body 2 having twenty-five microneedles 1 at the pitch of 1 mm, the microneedles 1 being made of polycarbonate and having a shape of a truncated cone with 100 μm in height, φ200 μm in base diameter and 50 μm in tip diameter.

The device body 5 was fixed with an adhesive at the center portion of the extension piece 36 of 60 mm×60 mm. The extension piece 36 was connected to one side of the positioning sheet 29 having the opening 35 of 20 mm×20 mm at the center portion of the quadrilateral of 60 mm×60 mm. The positioning sheet 29 and the extension piece 36 were made of a polyester film with the thickness of 10 μm. In addition, the pressure sensitive adhesive layers 54 and 56 were positioned on respective sides of the positioning sheet 29. Further, the peeling sheet of 50 mm×50 mm were connected to the rest of three sides of the positioning sheet 29 with the folding sections 50 of 10 mm×20 mm interposed therebetween. As the pressure sensitive adhesive layer 6a of the peeling sheet, an acrylic pressure sensitive adhesive (10 N/25 mm in adhesion) manufactured by Lintec Corporation was used. The substrate sheet 21 of the peeling sheet and the folding sections 50 were made of a polyester film with the thickness of 10 μm.

Using the transdermal administration device, the stratum corneum was removed by the peeling sheet. Medical fluids containing 1 mg, 10 mg and 20 mg of histamine (manufactured by Sigma) per 1 g of hydrophilic ointment were transdermally administered to the skin. In each of the cases, hives were developed. Thus, it was verified that the needles of the microneedles 1 certainly passed through the stratum corneum and reached the live cell layer (epidermis below the prickle cell layer) of the skin.

1. A transdermal administration device, comprising:
a device body including a needle form body, which includes a plurality of microneedles formed on a surface thereof and is provided with a fluid passage hole through the front surface to a back surface thereof, and a medical fluid filling section for providing a medical fluid to the back surface of the needle form body;
a stratum corneum peeling system; and
a connection section for connecting the device body and the stratum corneum peeling system,
wherein the stratum corneum peeling system is connected to the device body by the connection section such that the microneedles of the needle form body of the device body are positioned in an area of a skin surface in which a stratum corneum is peeled by the stratum corneum peeling system.

2. A transdermal administration device according to claim 1, wherein the device body includes: a cylinder body in which a syringe filled with the medical fluid is movably inserted; and the needle form body attached to a tip portion of the syringe.

3. A transdermal administration device according to claim 2, wherein a skin enlarging section for enlarging the skin when the cylinder body is pressed against the skin, is provided at a lower end opening of the cylinder body.

4. A transdermal administration device according to claim 1, wherein:
the device body includes: a base sheet; a capsule-shaped medical fluid filling section provided on one surface of the base sheet; and the needle form body provided on the other surface of the base sheet; and
a fragile portion is formed in the base sheet, the fragile portion being breakable by a pressure created by pressing the medical fluid filling section.

5. A transdermal administration device according to claim 1, wherein:
the connection section includes a supporting sheet provided on the side of the needle form body of the device body;
the supporting sheet includes an opening formed therein, the opening corresponding to the needle form body; and
the stratum corneum peeling system is connected to the device body with the supporting sheet interposed therebetween.

6. A transdermal administration device according to claim 5, wherein the connection section further includes a tearing sheet provided between the supporting sheet and the stratum corneum peeling system, and the tearing sheet can be torn along the skin surface so that it becomes possible to simultaneously tear the tearing sheet and the stratum corneum peeling system.

7. A transdermal administration device according to claim 6, wherein:
the tearing sheet is constituted of: a first sheet positioned in the middle; and second and third sheets respectively positioned on both sides of the first sheet;
the first sheet is adhered to end portions of the other two sheets, the end portions being folded; and
the second sheet is connected to the supporting sheet and the third sheet is connected to a substrate sheet of the stratum corneum peeling system.

8. A transdermal administration device according to claim 6, wherein:
the tearing sheet is constituted of: a second sheet with an end portion being folded; and a third sheet with an end portion being folded;
a folding section of the second sheet is connected to the supporting sheet; and
a folding section of the third sheet also serves as a substrate sheet of the stratum corneum peeling system.

9. A transdermal administration device according to claim 6, wherein:
the tearing sheet includes: a first sheet positioned in the middle; and second and third sheets respectively positioned on both sides of the first sheet;
an end portion of each of the sheets is folded;
the first, second and third sheets are adhered to the other sheets at the folded portions; and
the third sheet is connected to the stratum corneum peeling system.

10. A transdermal administration device according to claim 6, wherein:
the tearing sheet includes: a long-length sheet; and a short-length sheet;
the long-length sheet is folded and the folded portion is adhered with one side of the folded short-length sheet; and
the other side of the short-length sheet is connected to the stratum corneum peeling system.

11. A transdermal administration device according to claim 5, wherein a pressure sensitive adhesive layer is provided at least at a part of a side contacting the skin of the supporting sheet.

12. A transdermal administration device according to claim 1, wherein the stratum corneum peeling system includes: a substrate sheet; and a pressure sensitive adhesive layer laminated on the substrate sheet, and the stratum corneum on the skin surface can be peeled by the pressure sensitive adhesive layer.

13. A transdermal administration device, comprising:
a device body including a needle form body, which includes a plurality of microneedles formed on a surface thereof and is provided with a fluid passage hole through the front surface to a back surface thereof, and a medical fluid filling section for providing a medical fluid to a back surface of the needle form body;
a stratum corneum peeling system including: a substrate sheet; and a pressure sensitive adhesive layer laminated on the substrate sheet, the pressure sensitive adhesive layer peeling off a stratum corneum of a skin surface; and
a positioning sheet for connecting the device body and the stratum corneum peeling system,
wherein the positioning sheet includes an opening, the device body is connected to one end portion of the positioning sheet such that the device body can be folded and piled on the positioning sheet, and the stratum corneum peeling system is connected to the other end portion of the positioning sheet such that the stratum corneum peeling system can be folded and piled on the positioning sheet, and
wherein the microneedles of the needle form body are configured to be positioned at the opening when the device body is folded and piled on the positioning sheet, and the pressure sensitive adhesive layer of the stratum corneum peeling system is configured to be positioned at the opening when the stratum corneum peeling system is folded and piled on the positioning sheet.

14. A transdermal administration device according to claim 13, wherein the positioning sheet is formed in a quadrilateral shape in a planar view, the device body is connected to one side of the positioning sheet, and the stratum corneum peeling system is connected to the other side of the positioning sheet.

15. A transdermal administration device according to claim 13, wherein the positioning sheet is formed in a quadrilateral shape in a planar view, the device body is connected to one side of the positioning sheet, and the stratum corneum peeling system is connected to each of the other three sides of the positioning sheet.

16. A transdermal administration device according to claim 13, wherein an extension piece is extended from the positioning sheet, and the device body is adhered on the extension piece.

17. A transdermal administration device according to claim 13, wherein:
an extension piece is extended from the positioning sheet;
the device body includes: the extension piece; the capsule-shaped medical fluid filling section provided on one surface of the extension piece; and the needle form body provided on the other surface of the extension piece; and
the extension piece is provided with a fragile portion formed therein, the fragile portion being breakable by a pressure created by pressing the medical fluid filling section.

18. A transdermal administration device according to claim 13, wherein:
the device body includes: a medical fluid filling section for housing the medical fluid; and the needle form body provided on the exterior surface of the medical fluid filling section;
the plurality of microneedles are formed on the external surface of the needle form body;
the fluid passage hole is provided in the needle form body through the front surface to a back surface thereof; and
the fragile portion is provided in the medical fluid filling section, where the medical fluid filling section can be broken so that the medical fluid therein is provided to the back surface of the needle form body.

19. A transdermal administration device according to claim 13, wherein the pressure sensitive adhesive layer is provided at least at a part of a side contacting the skin of the positioning sheet.
